(19) United States
(12) Patent Application Publication
(10) Pub. No.: US 2007/0271122 A1
(43) Pub. Date: Nov. 22, 2007

(54) Patient Video and Audio Monitoring System

(75) Inventor: John R. Zaleski, West Brandywine, PA (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS MEDICAL SOLUTIONS USA, INC., MALVERN, PA (US)

(21) Appl. No.: 11/743,268

(22) Filed: May 2, 2007

Related U.S. Application Data
(60) Provisional application No. 60/796,775, filed on May 2, 2006.
(60) Provisional application No. 60/747,095, filed on May 12, 2006.

(51) Int. Cl. G06F 19/00; A61B 5/00

(52) U.S. Cl. 705/3

(57) ABSTRACT

A user interface and software system and architecture enables linking medical record information and patient location to a camera associated with that location when a particular patient is selected by a clinician using a health information system. The image of the patient as recorded by an in-room camera is displayed within a picture-in-picture display on a large plasma screen located at a command station, for example. The image may be controlled by a remote camera controller employing a Web-based user interface that allows a user to implement a pan-tilt-zoom (PTZ) camera controller via mouse control, for example. Information relating to patient location is used to remotely control, via Web-based application, the selection of the appropriate camera and the type of display in which to show the image. The user interface associated with this architecture provides an efficient and intuitive means of selecting specific images and specific display styles. The system operates in a parent-child relationship in which information associated with a parent application (HIS, for instance) is passed to the application architecture, thereby providing a direct, error-free and positive link to the application.

The present patent application derives priority from U.S. Provisional Patent Application Serial Number 60/796,775 by J. Zaleski filed May 2, 2006 and U.S. Provisional Patent Application Ser. No. 60/747,095 by J. Zaleski filed May 12, 2006.

### FIELD OF THE INVENTION

The present invention relates generally to the field of patient monitoring, and more specifically to a system for selecting and viewing audio-video data in combination with other data over a network.

### BACKGROUND OF THE INVENTION

In many industries a plurality of systems exist that enable users to monitor the actions of at least one other person by controlling the operation of a camera able to capture audio and video data representative of at least one person. Systems such as these are advantageous in the security and gaming industries as there is a need to continually monitor people and locations concurrently with other data. However, audio-visual monitoring systems are also advantageous in the healthcare industry and enable a physician or other healthcare provider to remotely monitor via real-time audio and video at least one patient. Known systems fail to address the problem of processing patient (and other context) information using a Web-based application involved in processing a camera image of at least one patient or multiple patients concurrently and related patient data using an optical switch and IP networking architecture. Known systems fail to enable web-based cohesive automatic switching from patient to patient as alerts and warnings occur. Known systems are reliant on user skill to manually manipulate navigation among patient images.

Known systems are unable to utilize a virtual interface for selection and control of cameras of the monitoring system. Virtual user interfaces (gaming, for instance) are normally designed for local operation at a single site (e.g.: a command center) with a hardware controller and lack the capability for remote concurrent operation at a plurality of different sites. Further, known systems are limited in the manner of function of pan, tilt, and zoom camera functions within the user interface, Specifically, known systems typically employ a camera controller that operates by dragging a mouse or controller stick within a pad window of a user interface using a dedicated hardware appliance. Such appliances typically employ proportional control: the distance the mouse is dragged from the origin, the larger the deflection in the camera. Proportional control is achieved in this way. The camera control and user interface of known systems is limited. A system according to invention principles addresses these deficiencies and associated problems.

### BRIEF SUMMARY OF THE INVENTION

A remote patient monitoring system according to invention principles enables visual monitoring of a plurality of patients in different locations concurrently from multiple (e.g., mobile) monitoring systems at a plurality different remote locations. An input processor receives a patient identifier derived from user interaction with an executable application. At least one repository of mapping information associates patient identifiers with corresponding camera identifiers and room identifiers enabling acquisition of video information from a location associated with a particular patient. A data processor uses the mapping information to automatically associate a received particular patient identifier with a particular camera identifier and to acquire video image data and audio concerning the particular patient from the particular camera, in response to user selection of a displayed image element associated with the particular patient in a display image associated the executable application. A display processor initiates generation of data representing a composite display image presenting the video image data of the particular patient in a first image window together with medical information concerning the particular patient in a second image window.

In a second embodiment, a system according to invention principles provides a remote patient monitoring system for visually monitoring patients in a plurality of different remote locations. An input processor receives a patient identifier derived from user interaction with an executable application. At least one repository of mapping information associates patient identifiers with corresponding camera identifiers and room identifiers enabling acquisition of video information from a location associated with a particular patient. A data processor uses the mapping information for automatically associating a received particular patient identifier with a particular camera identifier and initiating acquisition of video image data of the particular patient from the particular camera and initiates acquisition of medical parameter data from a monitoring device coupled to the particular patient, in response to user selection of a displayed image element associated with the particular patient in a display image associated with the executable application. A display processor initiates generation of data representing a composite display image presenting the video image data of the particular patient in a first image window together with the acquired medical parameter data, concerning the particular patient in a second image window. The data processor may automatically store the video image data and audio from the particular patient in a data repository record associated with the particular patient in response to a predetermined condition.

In a further embodiment according to invention principles, a remote patient monitoring system is provided for visually monitoring patients in a plurality of different remote locations. An input processor receives a patient identifier automatically provided as context information by an executable application. At least one repository of mapping information associating patient identifiers with corresponding camera identifiers and room identifiers enables acquisition of video information from a location associated with a particular patient. A data processor uses the mapping information for automatically associating a received particular patient identifier with a particular camera identifier and initiating acquisition of video image data of the particular patient from the particular camera, in response to user selection of a displayed image element associated with the particular patient in a display image associated with the executable application. A display processor initiates generation of data representing a composite display image presenting the video image data of the particular patient in a first image window together with medical information concerning the particular patient in a second image window.

A system according to invention principles provides a remote patient monitoring system for visually monitoring patients in a plurality of different remote locations. An input processor receives a patient identifier derived from user interaction with an executable application. At least one repository of mapping information associates patient identifiers with corresponding camera identifiers and room identifiers enabling acquisition of video information from a location associated with a particular patient. A display processor initiates generation of data representing a two dimensional image display indicating first and second axes and a cursor. A camera controller uses the mapping information for automatically associating a received particular patient identifier with a particular camera identifier and remotely initiating movement of a camera associated with the camera identifier in pan and tilt axes in proportion to cursor distance from an origin formed by the first and second axes of the two dimensional image.

### BRIEF DESCRIPTION OF THE DRAWING

FIG. 1 is a block diagram of the system for remote patient monitoring according to invention principles;

FIG. 2 is a flow diagram illustrating video data flow throughout the system for remote patient monitoring shown in FIG. 1 according to invention principles;

FIG. 3 is an illustrative view of the connection between elements of the system for acquiring patient video data according to invention principles;

FIGS. 4A-4F are illustrative views of different composite image display formats able to be displayed by the system according to invention principles;

FIG. 5 is a screen shot of a composite image display format displaying acquired video data in one of the individual data display windows according to invention principles;

FIG. 6 is an event trace diagram detailing operation of the remote patient monitoring system according to invention principles;

FIG. 7 is an exemplary display image of a boot page of the remote patient monitoring system according to invention principles;

FIG. 8 is HTML code representing an exemplary application launching method shown in FIG. 7 according to invention principles;

FIG. 9 is visual basic code representing an exemplary camera to patient mapping and association application according to invention principles;

FIG. 10 is an exemplary display image of the input/output channel selection utility according to invention principles;

FIG. 11 is an exemplary display image showing the input assigned using the utility of FIG. 10 according to invention principles;

FIG. 12 is an exemplary display image of the display format selection utility for selecting the format of the composite image to be displayed by the system according to invention principles;

FIG. 13 is an exemplary display image of the camera control interface utility for controlling camera operation and movement according to invention principles;

FIG. 14 is a detailed view of the camera control interface of FIG. 13 showing exemplary operation thereof according to invention principles; and

FIG. 15 is an illustrative view of the connection between elements of the system for controlling camera function and operation according to invention principles.

### DETAILED DESCRIPTION OF THE INVENTION

A processor, as used herein, operates under the control of an executable application to (a) receive information from an input information device, (h) process the information by manipulating, analyzing, modifying, converting and/or transmitting the information, and/or (c) route the information to an output information device. A processor may use, or comprise the capabilities of, a controller or microprocessor, for example. The use of the term manager or controller, as used herein may be synonymous with the term processor. The processor may operate with a display processor or generator. A display processor or generator is a known element for generating signals representing display images or portions thereof. A processor, manager and a display processor comprises any combination of hardware, firmware, and/or software.

An executable application, as used herein, comprises code or machine readable instructions for conditioning the processor to implement predetermined functions, such as those of an operating system, software development planning and management system or other information processing system, for example, in response to user command or input. An executable procedure is a segment of code or machine readable instruction, sub-routine, or other distinct section of code or portion of an executable application for performing one or more particular processes. These processes may include receiving input data and/or parameters, performing operations on received input data and/or performing functions in response to received input parameters, and providing resulting output data and/or parameters.

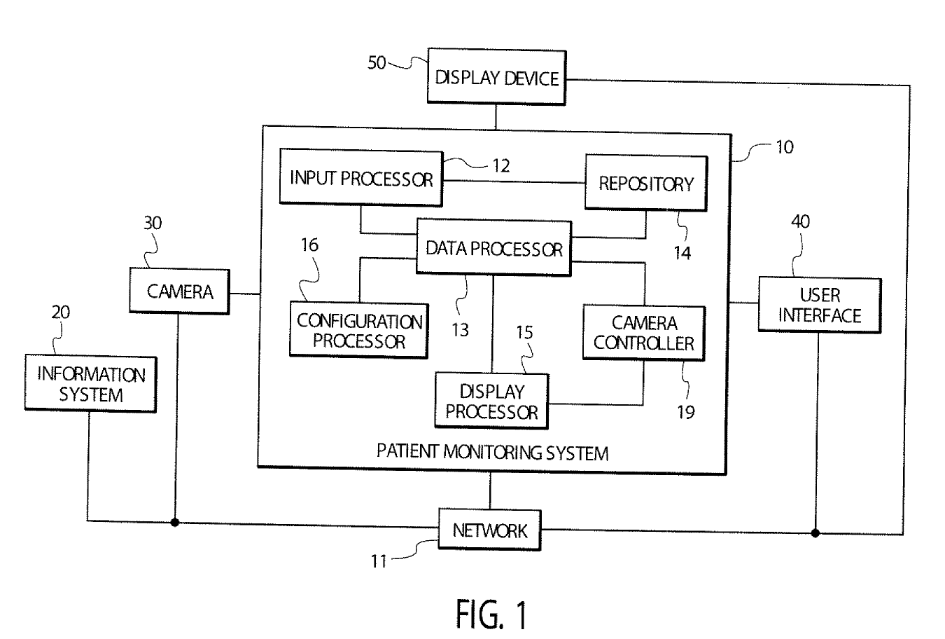

A user interface (UI), as used herein, comprises one or more display images, generated by the display processor under the control of the processor. The UI also includes an executable procedure or executable application. The executable procedure or executable application conditions the display processor to generate signals representing the UI display images. These signals are supplied to a display device which displays the image for viewing by the user. The executable procedure or executable application further receives signals from user input devices, such as a keyboard, mouse, light pen, touch screen or any other means allowing a user to provide data to the processor. The processor, under control of the executable procedure or executable application manipulates the UI display images in response to the signals received from the input devices. In this way, the user interacts with the display image using the input devices, enabling user interaction with the processor or other device. The steps and functions performed by the systems and processes of FIGS. 1-11 may be performed wholly or partially automatically or in response to user command.

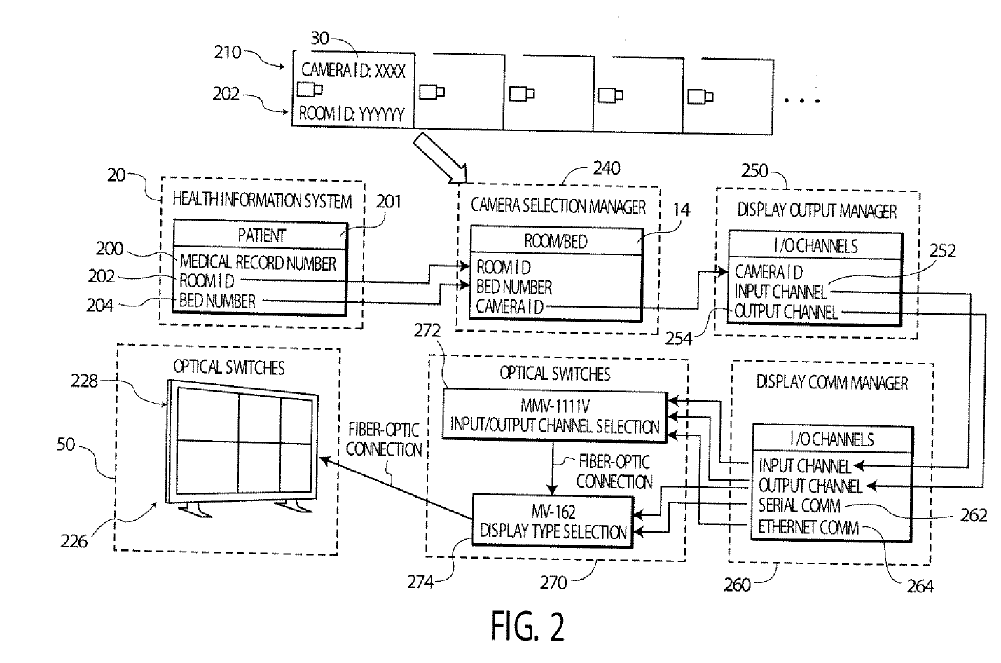

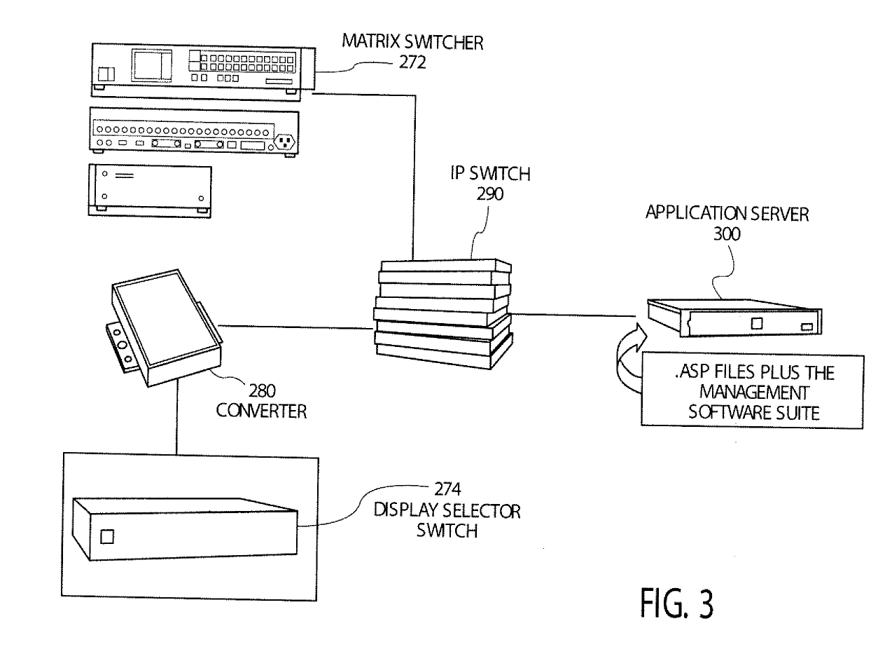

A patient monitoring system 10 (FIG. 1) facilitates audio-visual monitoring of a patient and enables a user, charged with the task of monitoring, to select a patient from a patient census list that is stored in a patient data repository. The system advantageously enables the viewing of a plurality of patients in different locations concurrently from multiple (e.g., mobile) monitoring systems at a plurality different remote. System 10 automatically provides a patient identifier that may be derived, in part from at least one of an alert, an order, patient context data or other clinical indicator. System 10 automatically maps the patient identifier and a room identifier maintained within an existing known repository (commonly referred to as a Master File) with a camera identifier that corresponds to a camera in the area of the patient and supports audio and visual data capture and transmission for display on a display device. The room identifier used herein identifies any area designated for patient care and may include, but is not limited to, a patient room, an operating room a bed location, a clinical care room, a trauma room, etc. The system automatically displays the patient video and audio data in a default image (FIGS. 2 and 3) that is at least one automatically determined in response to predetermined condition and selected in response to user command. The system 10 further supports automatic and/or selective storing of the video and audio data of the monitored patient in the particular patient record. The automatic storage of patient audio and video data may occur in response to predetermined criteria (e.g. an alert condition, an image detected irregular action, clinician command etc).

The patient monitoring system 10 resolves problems associated with passing and processing patient information using a Web-based application that also enables selecting a camera image using an optical switch and IP networking architecture to show the image of patient within a display with other patient data. Known systems employ IP Cameras (non fiber-based cameras and optical switches) and do not employ parent-child context communication nor a user interface described hereinbelow for controlling system 10. The system provides an improved workflow that aids clinicians who monitor many patients from a plurality of monitoring implementations. A patient is selected from a healthcare information system through a displayed patient census list and a patient record corresponding to the selected patient is displayed within a user interface. The patient record includes a plurality of hyperlinks that correspond to a camera trained on the selected patient and to various patient vital data or other patient information contained within the patient record. A patient identifier is communicated to system 10, which associates patient identifying information with video views and vitals views of the patient for display in a composite image. System 10 enables selection of different display formats for the displayed composite image to enable at least one of zooming in or out on the patient and display of patient video images and patient vitals parameters. The images and/or windows within the composite display image are selectively re-configurable in response to user command. Upon completion of the monitoring session, the user interface returns the user to a display of the patient census whereby a new patient may be selected and the process may begin anew. The system 10 and operation thereof is described hereinafter with respect to FIGS. 1-16. System 10 enables multiple users to view selected patients concurrently. The ability to concurrently view patients by multiple care providers is potentially restricted by network bandwidth limitations.

A block diagram of the patient monitoring system 10 is shown in FIG. 1. The system 10 may be connected via a communications network 11 to and communicate with any of a hospital information system 20, at least one camera or other audio-visual recording device 30, a user interface 40 and at least one display device 50. Communication between the system 10 and any device connected thereto may occur in any of a plurality data formats including, without limitation, an RS232 protocol, an Ethernet protocol, a Medical Interface Bus (MIB) compatible protocol, DICOM protocol, an Internet Protocol (I.P.) data format, a local area network (LAN) protocol, a wide area network (WAN) protocol, an IEEE bus compatible protocol, and a Health Level Seven (HL7) protocol, HTTP and HTTPS. Network communication paths may be formed as a wired or wireless (W/WL) connection. The wireless connection permits a user employing system 10 to be mobile beyond the distance permitted with a wired connection. The communication network 11 may comprise the Internet interconnecting a plurality of healthcare facilities or an Intranet connecting a plurality of departments of a particular healthcare organization. Additionally, while elements described herein are separate, it is well known that they may be present in a single device or in multiple devices in any combination. For example, as shown in FIG. 1, the user interface 40 and/or the recording device 30 may be connected directly to the monitoring system 10 without the use of a communications network.

The remote patient monitoring system 10 visually monitors patients in a plurality of different remote locations. The system 10 includes an input processor 12 for receiving and parsing data from an executable application residing on a remote information system 20 such as the healthcare information system (HIS) or a clinical information system (CIS). The information system 20 may include either the HIS or the CIS or any combination thereof. The at least one executable application, in response to at least one of a user request and a predetermined condition, automatically communicates a patient identifier that is derived from a patient medical record and which is unique to the particular patient. At least one data repository 14 is connected to the input processor 12 and includes data representing mapping information corresponding to camera identifiers and room identifiers for use in associating the patient identifier received by the input processor with a corresponding camera identifier and room identifier. This association enables acquisition of audio-video data from the location associated with the particular patient. The at least one repository 14 may be incorporated in system 10 or may be separate and accessed remotely via the communication network 11 in a known manner.

A data processor 13 is connected to the input processor 12 and the at least one repository 14. The data processor 13 uses the mapping information derived from the repository 14 and automatically associates a received particular patient identifier with a particular camera identifier. The data processor 13 may initiate acquisition of video image data of the particular patient from the particular camera 30 in the associated room or patient care area. The acquisition by the data processor 13 may occur in response to user selection of a displayed image element associated with the particular patient in a display image that is associated with the executable application in the information system 20 or patient record repository. The data processor 13 may also initiate acquisition of the video data at least one of automatically and in response to satisfaction of predetermined condition such as an alarm indicator. For example, acquisition of video data may occur when at least one of a patient medical parameter exceeds a predetermined threshold or range, a combination of a plurality of patient medical parameters exceeding corresponding predetermined thresholds or ranges and user command. In addition to video data, the data processor 13 may initiate acquisition of audio data that corresponds to the video data of the particular patient for use in monitoring the patient. The data processor 13 may use mapping information for automatically associating a received particular patient identifier with a particular camera identifier and initiating acquisition of video image data of the particular patient from the particular camera. The data processor 13 further initiates the acquisition and retrieval of medical parameter data from a monitoring device coupled to the particular patient, in response to user selection of a displayed image element associated with the particular patient in a display image associated with the executable application. The data processor 13 may also initiate storage of the acquired audio and/or video data. The acquired audio and/or video data may be stored in a patient record of the particular patient and may be stored in response to user command or in response to a predetermined criterion such as an alarm.

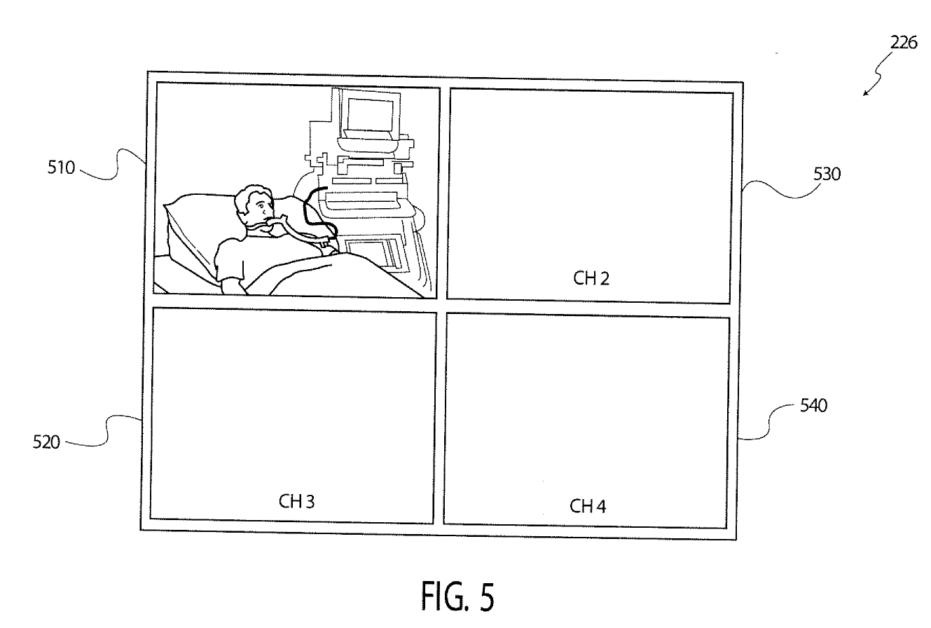

A display processor 15 is connected to the data processor 13 and initiates generation of data representing a composite display image, as shown in FIG. 5, presenting the acquired video image data of the particular patient in a first image window together with medical information concerning the particular patient in a second image window. The medical information displayed in the second window may be derived from information system 20 or a patient record stored in a patient record repository. Examples of medical information able to be displayed are waveforms, electronic patient charts, values obtained from medical devices connected to the patient such as ventilator parameter, ECG values, EEG values, etc. Any type of medical information corresponding to the particular patient of to a historical patient similarly situated may be displayed along with the video data of the patient. The video data and medical information displayed by system 10 may be displayed in a plurality of different user selectable windowing arrangements as will be discussed herein after with respect to FIG. 3. Thus, system 10 facilitates complete monitoring of patient condition and enables comparison of current patient data with information that a healthcare professional may deem useful. The composite display image generated by the display processor 15 may be displayed on a display device 50 that is local to system 10 or a display device 50 that is remotely located from system 10 via the communications network 11. Display processor 15 may generate data representing a composite display image that presents video image data of a plurality of different patients in a corresponding plurality of different image windows. A configuration processor 16 is connected to the display processor 15 and the data processor 13 and enables a user to selectively configure the composite display generated by the display processor 15 to concurrently display a video data plurality of different patients that may be in a single care unit in a healthcare facility in different windows within the composite display. Additionally, depending on the selected configuration (i.e. number and positioning of windows within the display), the configuration processor 16 is able to automatically configure the composite display to include medical information for the patient(s) being visually monitored.

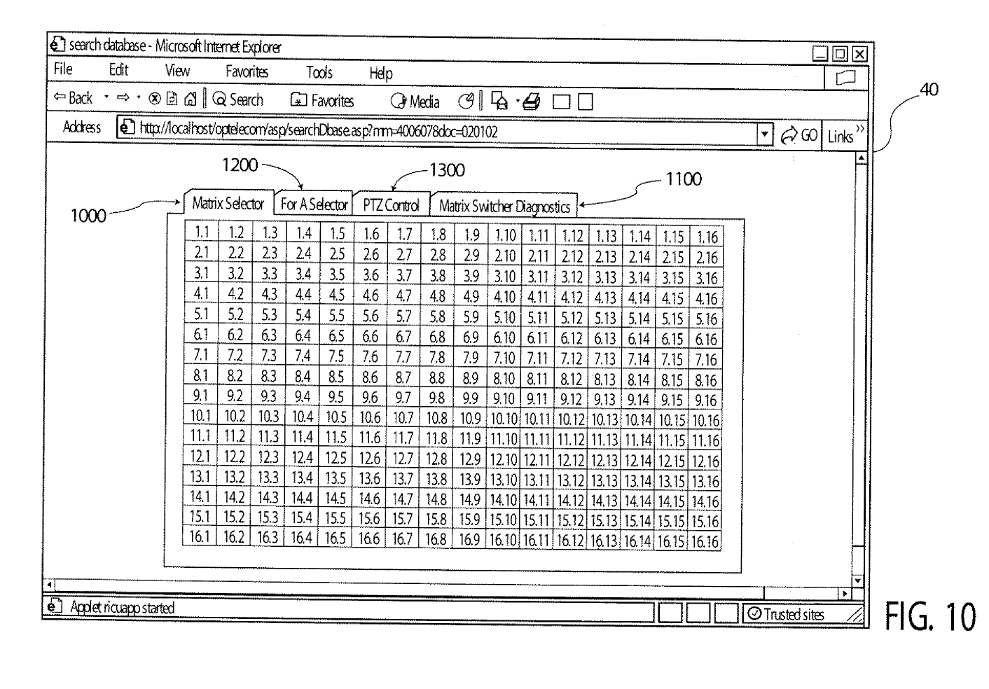

A user interface 40 enables a user to operate system 10 and also enables the user to control the particular camera 30 by controlling the pan, tilt and zoom of the camera 30 and enables real-time control of the camera 20 using a mouse-based interface and a virtual or real mouse pad. User interface 40 displays a display image including a plurality of image elements enabling a user to select and control system 10. For example, user interface 40 may display image elements representing particular patients that are derived from information system 20. User may select the image element representing a patient to begin video (and audio) monitoring of the patient. Other features of system 10 are fully enabled by user interface 40 and are discussed hereinafter with reference to FIGS. 10-14.

FIG. 2 is a functional flow diagram of patient monitoring system 10 showing the operation of system 10. Information System 20 (HIS or CIS) includes at least one executable application able to provide patient data from a patient record 201. Patient record 201 includes patient identifier 200, room identifier 202 and bed identifier 204. Once the patient medical record has been selected, a Web-enabled hyperlink communicates information corresponding to patient identifier 200 to a camera selection manager 240. Camera selection manager 240 performs similar functions as described hereinabove with respect to input processor 12 and data processor 13. In response to an initiating event, which includes but is not limited to, user selection of an image element representing the patient or occurrence of a predetermined condition (e.g. an alert), camera selection manager 240 receives patient identifier 200, room identifier 202 and bed identifier 204 from the information system 20 via a hyperlink generated in response to the initiating event. Camera selection manager 240 communicates with repository 14 which includes mapping information for mapping camera 30 to a particular patient identifier associated with a camera identifier 210, room identifier 202 and bed identifier using predetermined mapping information in repository 14. System 10 communicates with a plurality of cameras 30 enabling remote monitoring of a plurality of different patients at different locations throughout the healthcare facility. Camera selection manager 240 associates patient identifier 200 with camera identifier 210 and room identifier 202 and bed identifier 204 using predetermined mapping information to determine video (and audio) data to be acquired and monitored using a linkage between a fixed video camera 30 located in a patient's room for display in a selected window of a composite display 226 on a display device. Camera selection manager 240 associates a selected patient room and bed camera and communicates mapping data to display processor 15 (FIG. 1) and initiates acquisition of video data from the camera having the mapped camera identifier for generation of a display image including at least the acquired video data corresponding to the selected patient. Display processor 15 may include display output manager 250, display communication manager 260 and optical switches 270 as shown in FIG. 2. Upon receipt of mapping information from camera selection manager 240, display output manager 250 automatically determines the input channel 252 based on the camera identifier 210 associated with the particular camera for acquiring the video data from camera 30 of the particular patient and automatically assigns an output channel 254 to output the acquired video data to a composite display 226. The display output manager 250 is able to automatically acquire data on the input channel 252 and automatically assigns an output channel 254 according to user need and in such a manner to prevent conflict between channels when more than one patient is being monitored either in the same display 226 or using multiple display devices 50.

Display communication manager 260 facilitates transmission of video data acquired from camera 30 for display within a composite display image and further receives a control signal generated from system 10 specifying a window format of the composite display image including a number of individual display windows and defining the respective data output in each window. Display communication manager 260 receives video data provided by camera 30 via input channel 252 and outputs the received video data via an output channel to a first optical switch 272 able to control, select and route output channels for acquired video data using Ethernet (or LP Protocol) communication device 264. Once the video data is routed to a particular output channel via channel selection switch 272, the video data is provided to a display format selection switch which assigns the particular output video data to a predetermined individual data window within the composite display image. The routing of a video data acquired from a camera is discussed for exemplary purposes. It should be appreciated that system 10 may route multiple feeds of video data concurrently and also may acquire and control output of other medical data such as patient parameter data acquired from a device coupled to a particular patient.

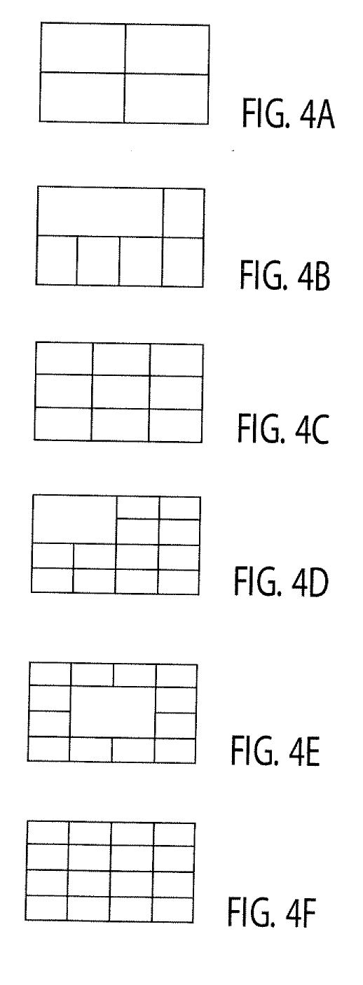

Display format selection is determined in response to a control signal received by display communication manager 260 that is at least one of automatically generated and generated in response to user command and provided to a second optical switch 274 enabling display format selection provided by serial communication unit 262. The video data and control signal may be output concurrently to their respective optical switches. The display communication manager 260 may be connected to the optical switches via coaxial or other known connection cable able to transmit audio and video data as shown in FIG. 3. The optical switches 272 may include an Ethernet-based matrix channel selection switch 272 for controlling and routing of multiple input and output channels of video data (e.g. a IDK MMV-1111V® marked and sold by IDK Technologies, Inc.) and a display selector switch 274 for controlling and selecting varying multi-window data viewing formats in a composite display (e.g. the FOR-A MV-162 marketed and sold by FOR-A®). The display selector switch 274 enables user selection of different multi-windowed displays formats for viewing at least the acquired patient video data as will be discussed in connection with FIG. 4. The operation of the format display switch may be automatic in response to a default composite image used by system 10 or automatically determined in response to a user preference profile or selectively controlled via software interface displayed within user interface 40 (FIG. 1), such as a web browser compatible user interface. Upon determination of the display format, the channel selection switch 272 provides the video data on the output channel assigned thereby to the display selector switch 274 which controls the display of the video data (with other data) in the specified window(s) 228 based on instructions of the control signal within composite display 226. Optical switches 270 translate the output received from the display communication manager 260 to an appropriate multi-view (e.g. picture-in-picture) composite display 226 on a display device 50 that may be located at least one of at a central command workstation or at a remote monitoring workstation accessible via a communications network.

System 10 may also selectively display other medical information data in different windows within the composite display 226. In response to the initiating event, data processor 13 (FIG. 1) may acquire medical information data from a plurality of different sources for display with the acquired video data. The other medical information data may be mapped by the data processor 13 in a manner similarly to the acquired video data and displayed on the display device. The display output manager 250 may determine which input and output channels 252, 254 are required for acquiring and outputting the other medical information data and the display communication manager 260 may control how the other medical information data is to be displayed in a similar manner as described above with respect to the acquired video data. Thus, patient monitoring system 10 advantageously automatically presents video and other medical information data in different windows of composite display 226. This enables a healthcare provider to have any relevant information regarding the particular patient available when monitoring the patient. Moreover, system 10 enables the healthcare provider to view medical information that may be stored in a facility remote from the particular healthcare facility such as a central patient record repository.

The connection between the optical switches 270 and the system 10 is shown in FIG. 3. A sever 300 may include a plurality of executable applications or applets for controlling system 10 operation. The sever 300 may also include an application enabling user selection and control of the optical switches 270. The server 300 is connected to the optical switches via an IP switch 290 that communicates between devices using a packet-based communication protocol such as internet protocol. The display selector switch 274 communicates with other devices using a serial bus and thus, a serial device server 280 such as the Nport 5110 by MOXA®, may be connected between the display selector switch 270 and the IP switch 290. The serial device server 280 facilitates remote management of, and communication with, the display selector switch 274 over an intranet. Server 300, connected on the same subnet as display selector switch 274 enables communication therebetween. An executable application is included on server 300 and, in conjunction with the monitoring application of system 10, converts and emulates he serial connection required for the display selector switch 274 over TCP/IP connection shown herein. Therefore, serial connectivity code may be connected to a virtual port that makes server 300 appear as if it is directly connected to the display selector switch 274 via a specific serial communications (COM) port and facilitates configuration of up to 256 COM ports. The channel selection matrix switch 272 communicates with server 300 using standard TCP/IP over Ethernet. Thus, the communication between applets on server 300 and the channel selector switch 272 occurs via TCP/IP and is addressable via standard socket communication.

FIGS. 4A-4F shows exemplary composite display formats that may be used by patient monitoring system 10. Display communication manager 260 (FIG. 2) may include display management application resident on server 300 (FIG. 3) and enable a user to selectively determine the format of the plurality of individual data windows 228 (FIG. 2) within the composite display 226 (FIG. 2). The plurality of different windows 228 within composite display image 226 are able to concurrently display data from different sources concerning different patients. The display format of system 10 may be set in a particular format as a default image format or may be automatically selected in response to a user preference profile of a specific healthcare provider who is authorized to operate patient monitoring system 10. Alternatively, the selection of the respective display format used by a particular healthcare provider is performed via image elements representing the respective format being generated and presented to a user for selection thereof. The implementation of the desired composite window display format is performed by the display selector switch which enables selection of the format and the channel selector switch which automatically enables locating a specific image in a specified window within composite display image 226.

FIG. 5 is an exemplary composite display image 226 in the format shown in FIG. 4A. The selected or determined composite display image 226 includes four individual data display windows 510, 520, 530 and 540. The first data display window 510 includes video data of a patient that has been selected by a user as discussed above. The data processor 13 associates the camera identifier with the patient identifier and acquired the video data of the particular patient. Display processor 15 commands the channel selection switch 272 (FIG. 2) to display the video data in first display window 510. Thus, the remaining three data display windows 520, 530 and 540 are free to display other different data such as medical information associated with the particular patient being visually monitored by the user. Specifically, because patient information is known (i.e., patient medical record number and room and bed location), other screens within the picture-in-picture display may be populated with patient specific information, such as bedside waveforms, charts, still images (x-rays, MRIs, etc., from picture archiving systems), and real-time video of the patient.

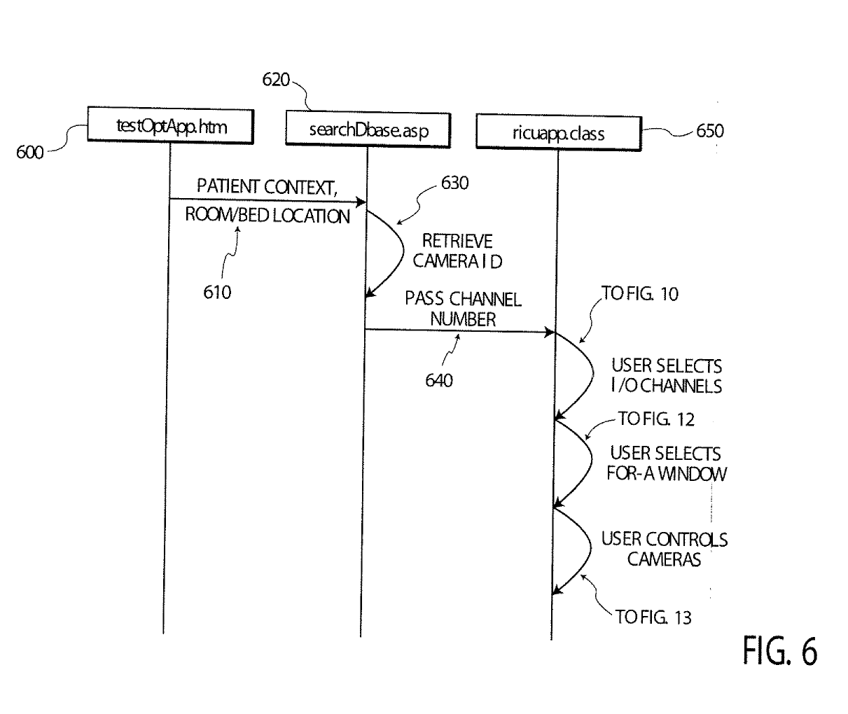

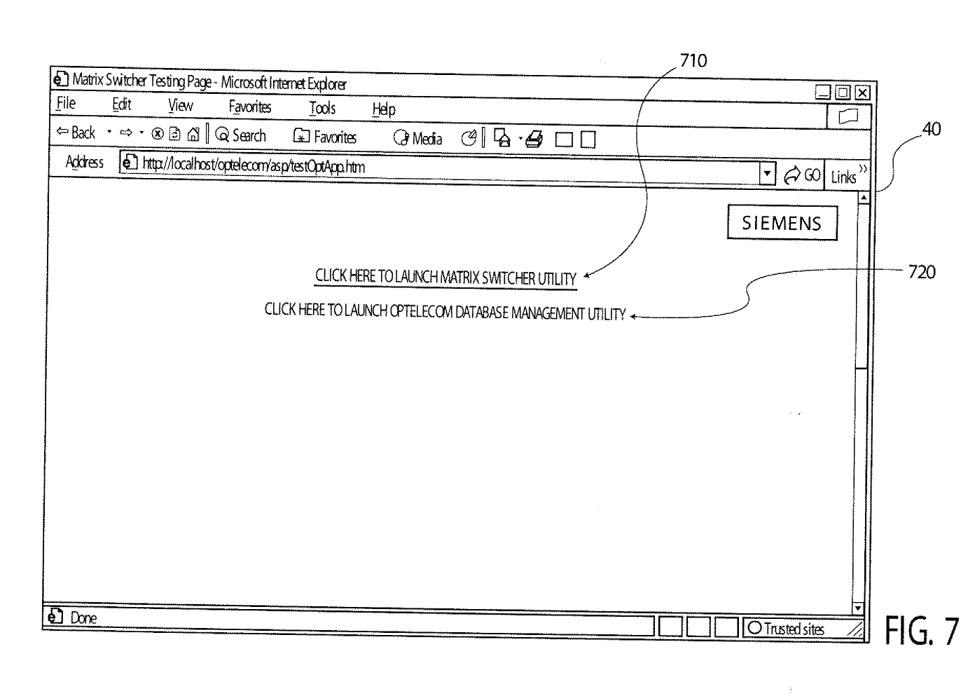

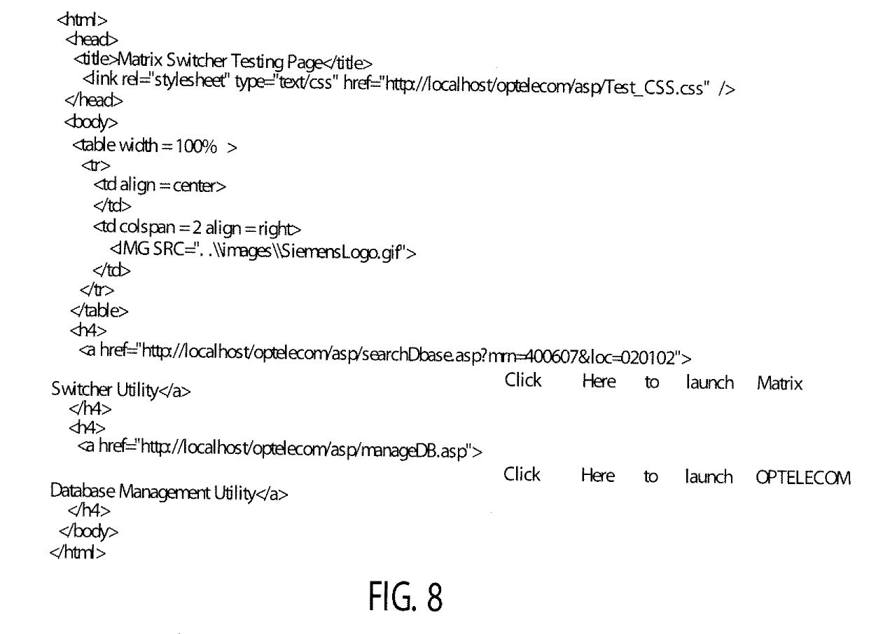

An Event Trace Diagram (ETD) is provided in FIG. 6 and illustrates the flow of data between the above described components of patient monitoring system 10. While described herein with reference to software applications, it should be appreciated that the functions of individual applications may be hard-coded as firmware on respective processors. A user initiates operation of system 10 via user interface 40 (FIG. 7) connected thereto. For purposes of example, the monitoring application is launched via an HTML webpage boot agent 600 entitled testOptApp.htm, the exemplary code for which is shown in FIG. 8. However, testOpApp.htm 600 may be any context-passing information system (e.g. healthcare information system or clinical information system) Web page enabling passing at least one of patient identifier, room identifier and bed identifier to monitoring system 10. Thus, the User interface 40, in FIG. 7, displays a plurality of hyperlinks 710 and 720 initiating operation of respective features for patient monitoring to be performed by system 10. Thus, the boot agent 600 (FIG. 6) is the parent Web-based application (health or clinical information system) for transmitting patient identifier (e.g. medical record number), room identifier (e.g. room number) and bed identifier via URL 610 to a mapping application 620 in the monitoring system 10.

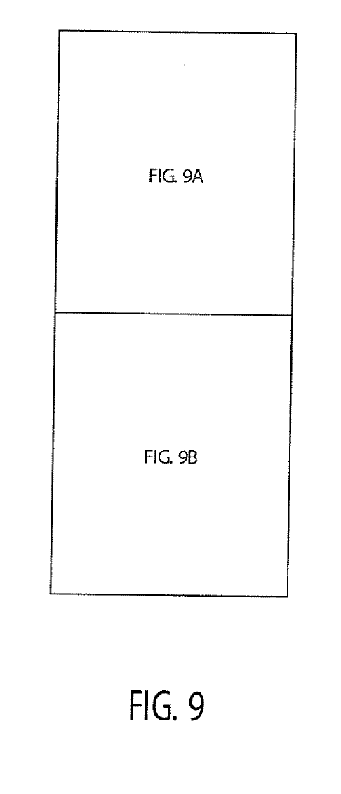

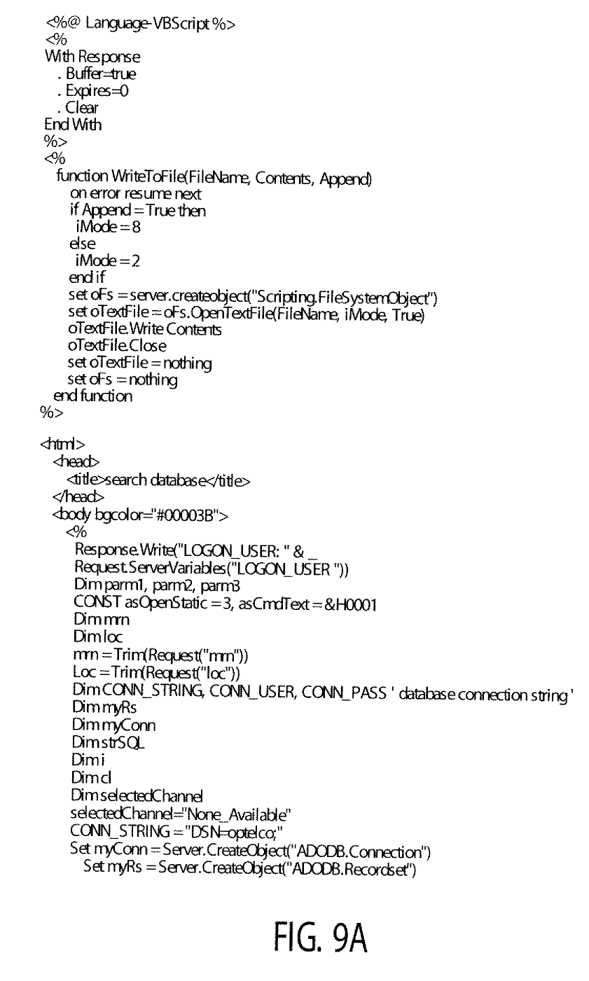

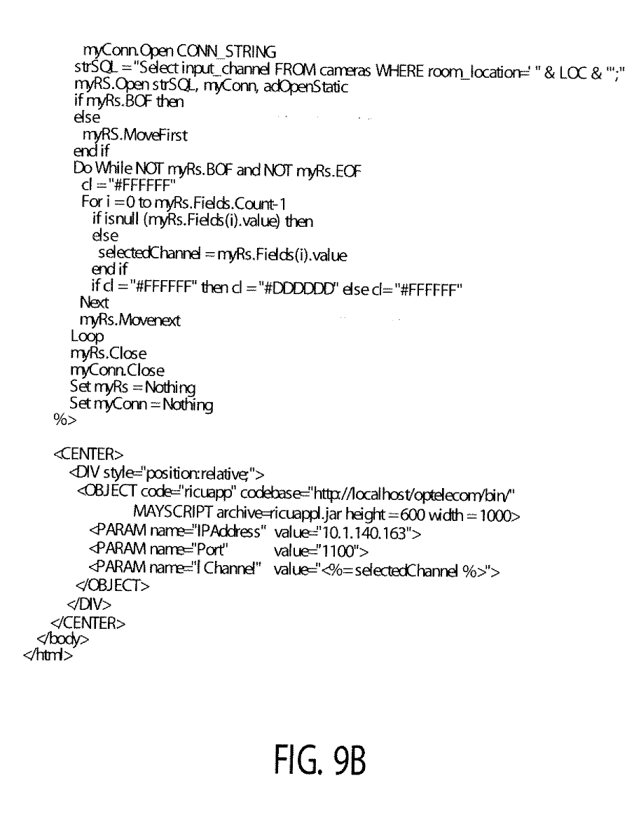

An exemplary mapping application 620 is represented as searchDbase.asp, the exemplary code for which is shown in FIG. 9. URL 610 containing the respective identifiers is passed by the boot page 600 to the mapping application 620 which queries a database table located in file optelco.mdb that includes pre-loaded mapping information. The mapping information associate camera identifiers corresponding to individual cameras to patient care locations such as a patient room. In one embodiment each camera is at a fixed location within each patient room. In another embodiment the camera may be in a mobile location e.g. a mobile camera in a care unit or on a mobile unit such as a mobile medical device cart. The system provides a specific mapping between each camera and each patient care location wherein at least one patient, having a patient identifier, is in a respective care location.

Patient monitoring system 10 automatically establishes a link 630 (FIG. 6) among a patient, a patient bed, a room, and a camera using the various identifiers. Specifically, using the room/bed identifier location parameters as a key from which to identify an associated input channel (i.e., for data from a camera) 640. An identifier of an input channel 640 is thereby returned and transmitted to the display control application 650 represented herein as a signed Java applet ricuapp.class. A signed java applet is used because of Java security restrictions on TCP/IP and serial connectivity. By using a signed (i.e., trusted) applet, communications with the channel selection switch 272 (FIG. 2) and the display selector switch 274 (FIG. 2) individual cameras are enabled to capture video data of the selected patient. The applet operation is illustrated in the user interface screen shots in FIGS. 10-13. The user interface 40 in FIGS. 10-12 include a plurality of tabbed application pages. Display control application includes mapping tab 1000, channel selection tab 1100, display format selection tab 1200 and camera control tab 1300.

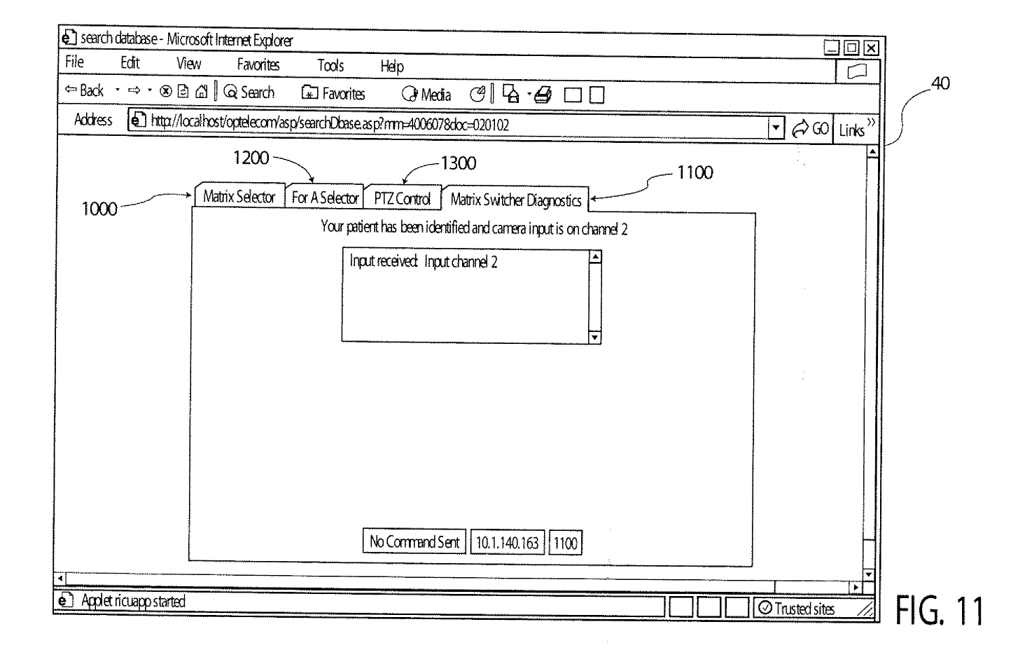

FIG. 10 is a screenshot of the user interface 40 having the mapping tab 1000 selected enabling selection of the input output channels. The mapping tab 1000 includes a matrix map of input channels (rows) to output channel (columns) for the screens display on a display device. The mapping of channels shown in FIG. 10 are fully user-selectable. Each image element may be a button within the matrix contains a label marked as “X, Y” where X represents the input channel and Y represents the output channel. The user may select output channels (i.e., screens) on which to display patient video data acquired from the respective camera. In this way, the user may add multiple input channels to output channels, and may chance the display at will. Thus, the user may depress the button marked “2,1” in the applet 650 (FIG. 6) and the image of the patient will be transferred from input channel “2” to output channel “1”. When a specific patient has been selected and link 630 (FIG. 6) is completed to produce a corresponding input channel 640, the input channel 640 may automatically be provided to the Java applet 650 and becomes the default input channel for video acquired by system 10 and utilized by applet 650. This assignment of a default video input channel is shown in FIG. 11 when the channel selection tab 1100 is active.

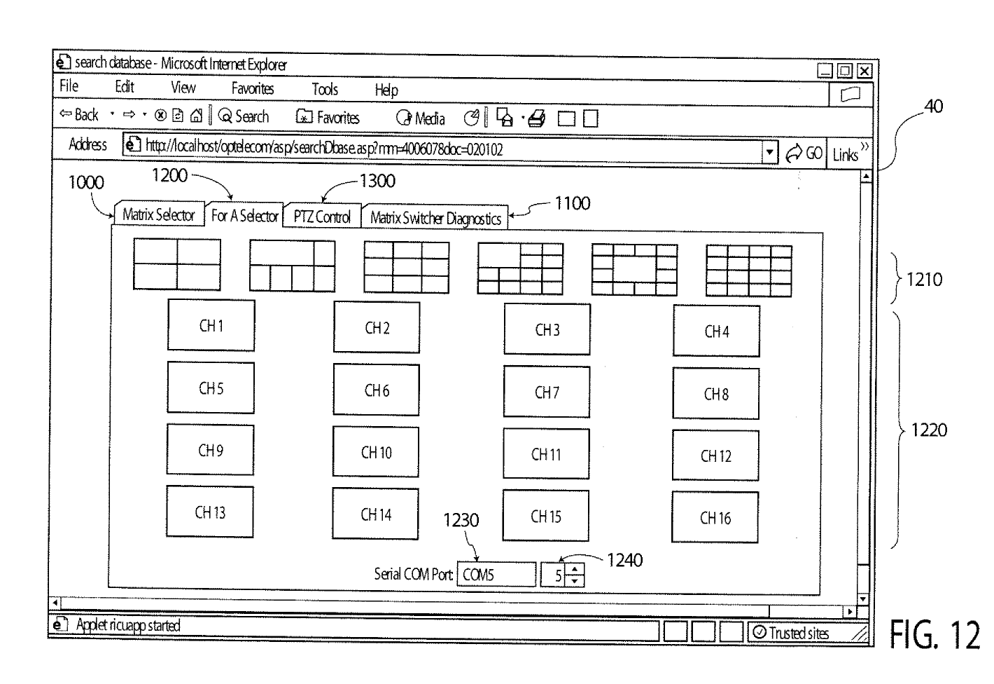

Upon selecting and assigning user input and output channels for the video data, a user may selective determine the format of the composite display image in which the video (and other data) is to be displayed. The format selection tab 1200 of display control application 650 is selected and shown in FIG. 12. Format selection tab 1200 includes a plurality of image elements 1210 representing different composite image display configurations for a particular patient. The composite image display configurations are formed from a plurality of individual data windows able to display data from different data sources being communicated over one of a plurality of output channels. The format selection tab 1200 further includes a plurality of image elements representing individual output channels 1220. A user may select the image element representing a composite image display and populate the individual windows of the composite display image with data being output over the output channels by assigning a particular output channel to a particular individual data display window. Assignment of output channels may be performed by at least one of selection of a channel and a respective one of a plurality of individual display windows and by dragging an image element 1220 representing a respective one of a output channel and dropping the selected output channel image element within an individual display window within the composite display image element 1210. Alternatively, the composite format display may be automatically selected by system 10 based on at least one of a predetermined default composite image display and a composite image display stored in a user preference profile associated with an authorized user of system 10. Additionally, as the format selection tab controls the operation of the format display switch 274 (FIG. 2) which communicates over an RS232 serial bus, the tab 1220 provides a COM port identification window 1230 indicating the current COM port selected for output of the acquired video image and an image element representing a COM port selector 1240. The COM port selector 1240 enables a user to selective determine the COM port being used by the format selection switch 274. As discussed above, the format selection switch 274 enables configuration of 256 COM ports and each COM port may be selectively configured by a user with unique format display configurations.

System 10 advantageously provides a user with an enhanced control interface allowing the user to selectively control the movement and view of the respective camera 30 (FIG. 1) from which the video (and audio) data is being acquired and monitored. For example, system 10 enables a user within a remote intensive care unit setting to access and control viewing cameras on each patient without the need for physical proximity to the camera control hardware. The interface 1300 maybe Web-based and may be an executable application designed as a thin-client object that may be loaded in a browser window. Camera control interface 1300 may exist independent from or in conjunction with the other interfaces 1000, 1100 and 1200.

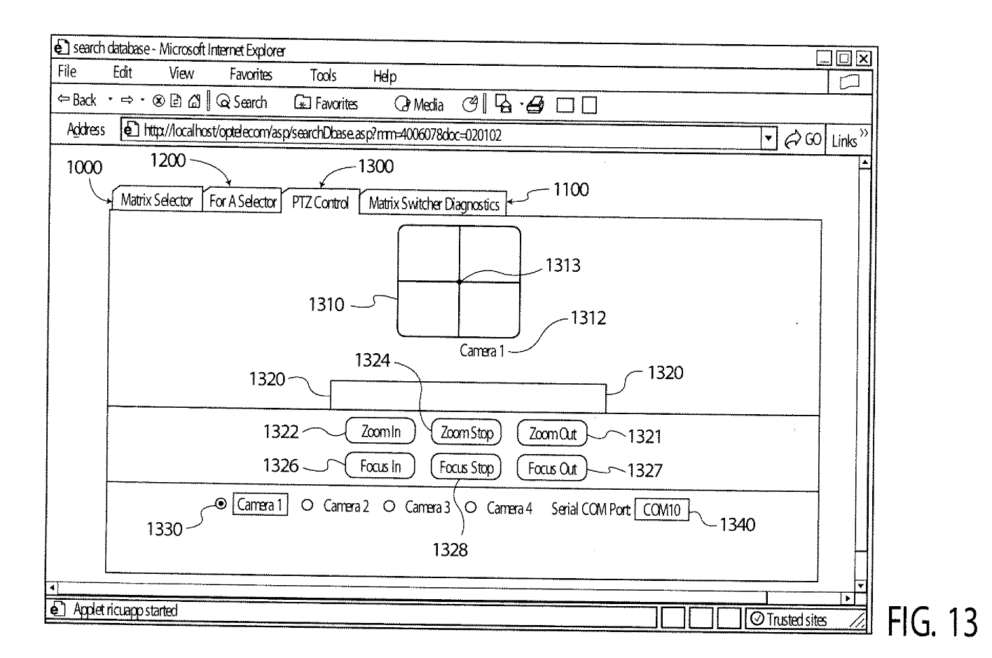

As shown in FIG. 13, user interface 40 is displaying a camera control interface tab 1300. Camera control interface tab 1300 advantageously enables remote operation of camera 30 that does not require the user to utilize the actual camera controller interface. The user interface enables real-time control of the camera 30 using a mouse-based interface and a virtual or real mouse pad. System 10 communicates a series of commands to a camera controller interface via communication network 11 (FIG. 1). Camera control interface tab 1300 enables transmission of data representing camera operation instructions to the controller interface by at least one of RS232 serial communication and serial communication via TCP/IP protocol using a serial device server 280 as described with respect to FIG. 3.

The camera control interface tab 1300 includes image elements representing a camera selector 1330 that may be generated by display processor 15 (FIG. 1). Camera selector 1330 enables a user to select the camera 30 that is operating on the serial COM port identified in COM port identification window 1340 to be controlled via an interface image associated with image tab 1300. Upon selection of camera 30, an identifier corresponding to the selected camera 30 is displayed in camera identifier window 1312. Positioned adjacent camera identifier window 1312 is a control image element 1310. Control image 1310 is a two dimensional image display indicating first and second axes forming a four quadrant display image and a position indicating cursor 1313, which when at rest, resides at the origin (0,0) of the first and second axes. User may use an input device (e.g. a mouse, light pen, touchpad, etc) to selectively control the movement of the selected camera. A user may select position indicator 1313 which may be a cursor and move the position indicator 1313 along the first and second axes of control image 1310. Selection and movement of position indicator 1313 causes a camera controller to generate movement instructions causing remote movement of the camera associated with the camera identifier. The distance of the position indicator 1313 (cursor) controls at least one a speed of camera movement and a camera angle. Movement instructions are generated in response to movement of the input device in direct proportion to the distance and direction that the position indicator 1313 is moved from the origin in any coordinate direction. Movement instructions may be a series of successive points along the first and second axes and are transmitted by camera controller 19 (FIG. 1). Camera motion may be stopped by releasing the selection button used to select and move position indicator 1313 resulting in the position indicator 1313 to return to the origin.

Camera control interface tab 1300 further includes a plurality of image elements 1320 enabling control of viewing features such as focus and zoom. Control interface 1300 includes a zoom in button 1322, a zoom out button 1321 and a stop zoom button 1324 which ceases operation of the zoom functionality of camera 30. The focus of the camera lens may be selectively adjusted in response to user selection of a focus in button 1326, focus out button 1327 and focus stop button 1328. System 10 advantageously enables remote initiation of pan and tilt axis camera movement in proportion to cursor distance from an origin formed by the first and second axis in camera control image 1310.

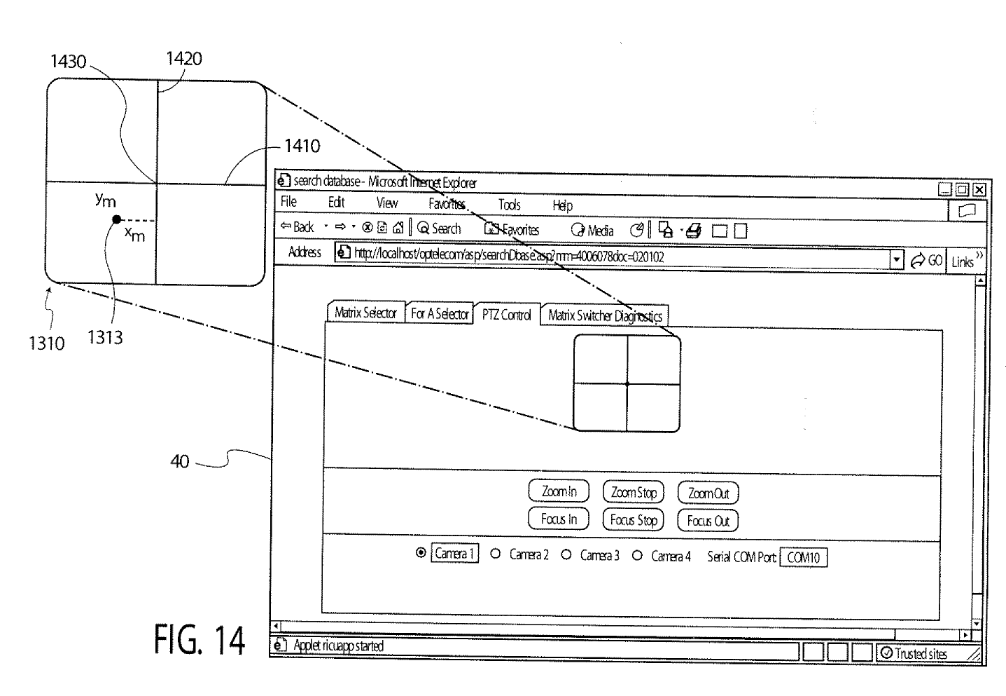

The remote movement of camera 30 is further depicted in FIG. 14 which shows an expanded view of control image 1310 on an exemplary camera control interface tab 1300. Control image 1310 includes first axis 1410 and second axis 1420 which bisects first axis 1410 forming a quadrant. For exemplary purposes, first axis 1410 is the X-axis and second axis 1420 is the Y-axis. Position indicator 1313 has been moved to position (Xm,Ym) resulting in camera 30 being caused to move to a point proportionally equivalent to position (Xm,Ym) from the origin 1430.

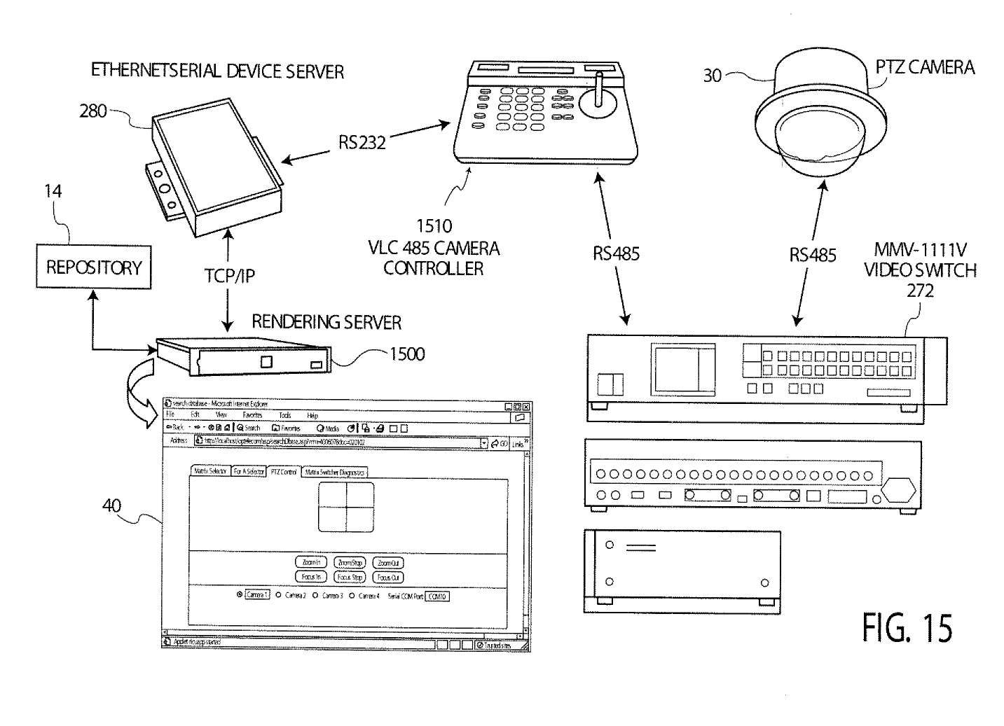

An exemplary hardware arrangement able to operate system 10 as discussed above in FIGS. 13 and 14 is shown in FIG. 15. Processor 1500 communicates using TCP/IP protocol and may include an input processor for receiving a patient identifier derived from user interaction with an executable application, a display processor for initiating generation of data representing the control image 1310 (FIG. 13) and a camera controller for controlling operation of camera 30. At least one repository is connected to processor 15 and includes mapping information associating patient identifiers with corresponding camera identifiers and room identifiers enabling acquisition of video information from a location associated with a particular patient. Processor 1500 is connected to user interface 40 and generates a camera control interface image such as shown in FIG. 13. For example, processor 1500 may be a server or rendering server which may be the source for Web-based content by rendering a Web-based user interface on a remote compute client that is proximal to a viewing screen on which any selected camera can be viewed. Processor 1500 is further connected via serial device server 280 to a cameral control apparatus 1510. Processor 1500 communicates via TCP/IP over Ethernet which is translated by serial device server 280 into RS232 standard communication which is used by camera control apparatus 1510. Camera control apparatus 1510 communicates to any camera 30 via RS485 wiring through a matrix switch 272 that supports multiple camera video feeds over single mode fiber. This RS485 control wiring extends to the remote camera in any location within a healthcare facility. Additionally, the RS485 connection between control apparatus 1510 and both camera 30 and matrix switch 272 may be wireless, wired or any combination thereof. Movement instructions corresponding to direction zoom, pan, tilt and focus that are generated by a user interacting with camera control image 1300 are transmitted to the camera via the above discussed connections and further provided to the switch 272 to be rendered on a display. As discussed, in addition to acquired video information, additional data such as medical data, may similarly transmitted to matrix switch 272 for display along with the acquired video information.

System 10 advantageously provides continuous and proportional control of camera operation such as smooth operation with respect to user-input device position as well as camera pan and tilt speed proportional to a distance of a position indicator from the origin of a camera control image. Known systems typically use Keypad type or fixed button control in which multiple clicks on a specific button translate into increased camera motion and distance. In contrast the system advantageously provides proportional control based on proximal location of a position indicator with respect to origin in pan and tilt coordinates that establish the location of the camera and the speed with which it moves. The system advantageously provides an ability to select any camera within one or more enterprises and adjust its features using a thin-client Web-based interface requiring no local software installation.

Although the preferred embodiments for the invention have been described and illustrated, the specific charts and user interfaces are exemplary only. Those having ordinary skill in the field of data processing will appreciate that many specific modifications may be made to the system described herein without departing from the scope of the claimed invention.

What is claimed is:

1. A remote patient monitoring system for visually monitoring patients in a plurality of different remote locations from a plurality of different locations, comprising:
an input processor for receiving a patient identifier derived from user interaction with an executable application;
at least one repository of mapping information associating patient identifiers with corresponding camera identifiers and room identifiers enabling acquisition of video information from a location associated with a particular patient;
a data processor for using said mapping information for automatically associating a received particular patient identifier with a particular camera identifier and initiating acquisition of video image data of said particular patient from said particular camera, in response to user selection of a displayed image element associated with said particular patient in a display image associated with said executable application and enabling the viewing of a plurality of patients in different locations concurrently from a plurality different remote locations; and
a display processor for initiating generation of data representing a composite display image presenting said video image data of said particular patient in a first image window together with medical information concerning said particular patient in a second image window.

2. A system according to claim 1, including
user interface enabling control of said camera pointing in pan, tilt, and zoom.

3. A system according to claim 2, wherein
said user interface enables real-time control of said camera using a mouse-based interface and a virtual or real mouse pad.

4. A system according to claim 1, wherein
said executable application is at least one of, (a) a hospital information system and (b) a clinical information system and
said display image associated with said executable application provides a list of different patients.

5. A system according to claim 1, wherein
said composite display image employs a default image format.

6. A system according to claim 1, wherein
said composite display image employs an image format determined by a user preference profile.

7. A system according to claim 1, wherein
said composite display image employs an image format selected by said user.

8. A system according to claim 1, wherein
said data processor initiates acquisition of audio data of said particular patient.

9. A system according to claim 1, wherein
said remote patient monitoring system is employs a Web browser compatible user interface.

10. A remote patient monitoring system for visually monitoring patients in a plurality of different remote locations, comprising:
an input processor for receiving a patient identifier derived from user interaction with an executable application;
at least one repository of mapping information associating patient identifiers with corresponding camera identifiers and room identifiers enabling acquisition of video information from a location associated with a particular patient;
a data processor for using said mapping information for automatically associating a received particular patient identifier with a particular camera identifier and initiating acquisition of video image data of said particular patient from said particular camera and initiating acquisition of medical parameter data from a monitoring device coupled to said particular patient, in response to user selection of a displayed image element associated with said particular patient in a display image associated with said executable application; and
a display processor for initiating generation of data representing a composite display image presenting said video image data of said particular patient in a first image window together with said acquired medical parameter data, concerning said particular patient in a second image window.

11. A system according to claim 10, wherein
said data processor automatically stores said video image data of said particular patient in a record associated with said particular patient in response to a predetermined condition.

12. A system according to claim 11, wherein
said predetermined condition comprises at least one of, (a) a patient medical parameter exceeding a predetermined threshold or ranges (b) a combination of a plurality of patient medical parameters exceeding corresponding predetermined thresholds or ranges and (c) user command.

13. A system according to claim 10, wherein
said display processor initiates generation of data representing a composite display image presenting video image data of a plurality of different patients in a corresponding plurality of different image windows.

14. A system according to claim 13, wherein
said plurality of different patients comprise patient assigned to a single care unit.

15. A system according to claim 13, including
a configuration processor enabling a user to graphically select multiple patient or single patient views for presentation in said composite display image.

16. A remote patient monitoring system for visually monitoring patients in a plurality of different remote locations, comprising:
an input processor for receiving a patient identifier automatically provided as context information by an executable application;
at least one repository of mapping information associating patient identifiers with corresponding camera identifiers and room identifiers enabling acquisition of video information from a location associated with a particular patient;
a data processor for using said mapping information for automatically associating a received particular patient identifier with a particular camera identifier and initiating acquisition of video image data of said particular patient from said particular camera, in response to user selection of a displayed image element associated with said particular patient in a display image associated with said executable application; and
a display processor for initiating generation of data representing a composite display image presenting said video image data of said particular patient in a first image window together with medical information concerning said particular patient in a second image window.

17. A system according to claim 16, wherein
said patient identifier is derived in response to user selection of a displayed image element associated with a particular patient identified in a list of a plurality of different patients provided by said executable application.

18. A remote patient monitoring system for visually monitoring patients in a plurality of different remote locations, comprising:
an input processor for receiving a patient identifier derived from user interaction with an executable application;
at least one repository of mapping information associating patient identifiers with corresponding camera identifiers and room identifiers enabling acquisition of video information from a location associated with a particular patient;
a display processor for initiating generation of data representing a two dimensional image display indicating first and second axes and a cursor; and
a camera controller for using said mapping information for automatically associating a received particular patient identifier with a particular camera identifier and remotely initiating movement of a camera associated with said camera identifier in pan and tilt axes in proportion to cursor distance from an origin formed by said first and second axes of said two dimensional image.

19. A system according to claim 18, wherein
said camera controller enables control of said camera pointing in pan, tilt, and zoom.

20. A system according to claim 18, wherein
said cursor distance from said origin controls at least one of, (a) speed of camera movement and (b) camera angle.

21. A system according to claim 18, wherein
said user interface enables real-time control of said camera using a mouse-based interface and a virtual or real mouse pad.

22. A system according to claim 18, including
a display processor for initiating generation of data representing a composite display image presenting video image data of said particular patient in a first image window together with medical information concerning said particular patient in a second image window.
